(19) United States
(12) Patent Application Publication
(10) Pub. No.: US 2005/0009893 A1
(43) Pub. Date: Jan. 13, 2005

(54) Treatment of high blood pressure during the acute phase of a stroke

(75) Inventor: Joachim Schrader, Cloppenburg (DE)

Correspondence Address: CROWELL & MORING LLP;INTELLECTUAL PROPERTY GROUP, P.O. BOX 14300, WASHINGTON, DC, 20044-4300, US

(73) Assignee: Solvay Pharmaceuticals GmbH, Hannover (DE)

(21) Appl. No.: 10/851,660

(22) Filed: May 24, 2004

Related U.S. Application Data
(63) Continuation of application No. PCT/EP02/13238, filed on Nov. 25, 2002.

(30) Foreign Application Priority Data
Nov. 23, 2001 (DE) 10157474.6
Nov. 27, 2001 (DE) 10158030.4

(51) Int. Cl. A61K 31/4184; A61K 31/401

(52) U.S. Cl. 514/381; 514/423

(57) ABSTRACT

A method of treating an acute cerebral vascular event (stroke) by administering a substance which inhibits the renin-angiotensin system.

### CROSS-REFERENCE TO RELATED APPLICATIONS

This application is a continuation of International Patent Application No. PCT/EP02/13238, filed Nov. 25, 2002 designating the United States of America, and published in German as WO 03/043615, the entire disclosure of which is incorporated herein by reference. Priority is claimed based on Federal Republic of Germany patent application nos. 101 57 474.6 and 101 58 030.4, filed Nov. 23 and Nov. 27, 2001, respectively.

### BACKGROUND OF THE INVENTION

The subject of the invention is the use of antihypertensive agents—especially substances which inhibit the rennin-angiotensin system (angiotensin-receptor-blockers, ACE-inhibitors, vasopeptidase-inhibitors) as well combinations of these substances and other antihypertensive agents—in the treatment of an acute stroke in order to reduce the risk cerebrovascular or cardiovascular events and to improve the neurological prognosis of the patient.

The treatment of high blood pressure in the acute therapy of a stroke has not previously been investigated.

Each year approximately 350,000 persons suffer a stroke in the Federal Republic of Germany. At least 70,000 of them die. Stroke thus represents the third most frequent cause of death in Germany. Many patients experience long-lasting and severe neurological consequences; only a small proportion ever become completely capable of working again. At the present time in Germany more than one million patients must live with long term disabilities as the consequence of strokes, which often lead to premature retirement from professional life and to social isolation. These numbers underscore the importance of coming to an understanding of the basis, the therapy, the pre-treatment and the post-treatment of a stroke. The most common causes of a stroke are ischemic brain infarct (brain hemorrhage). The rate of reoccurrence after a transitory ischemic attack amounts to 5% per year, after a manifest stroke 10% per year, and after a severe stroke 15% per year. Fifty to seventy-five percent of the affected patients eventually become somewhat independent again, but twenty-five percent require continuing nursing care.

The most important risk factor for a stroke is high blood pressure. While the importance of antihypertensive therapy in the primary prevention of a stroke is undisputed, the prevailing opinion is that such treatment is contraindicated in the acute phase of a stroke and in secondary prevention.

There is no doubt that antihypertensive therapy demonstrates its greatest success in primary prevention of a stroke. Taken together, all prevention studies show that a reduction of the diastolic blood pressure by 5 to 6 mmHg and of the systolic blood pressure by 10 to 12 mmHg reduces the frequency of a stroke by approximately 42%. Even in isolated systolic high blood pressure, antihypertensive therapy reduces the frequency of strokes by approximately one third. Similar success is also achieved in old patients with high pressure.

Also, after a completed stroke, i.e., during secondary prevention, antihypertensive therapy, which generally is commenced two weeks after the end of the stroke, reduces the risk of a new event. However, the data for such patients has not been investigated as thoroughly as the data for patients undergoing primary prevention therapy. Thus, a J-curve between blood pressure and the reappearance of strokes in patients undergoing secondary preventive treatments is discussed over and over. However, the previously available data does not give any certain evidence of the existence of a J-curve like relationship between blood pressure and the risk of a new apoplectic attack. In the evaluation of the UK-TIA trial, it could be shown in the cases of 2,435 patients that the reoccurrence of strokes also increased with the blood pressure level. A diastolic blood pressure which was lower by 5 mmHg was associated with a one third lower occurrence of strokes. This result thus is comparable with the studies of the incidence of a first stroke. The absolute difference in the incidence of strokes, which were associated with this type of blood pressure difference, was clearly higher than in studies of the frequency of primary strokes.

The treatment of high blood pressure in acute therapy of strokes has, as yet, not been investigated.

In order to clarify the question, the ACCESS study (acute candesarten cilexetil evaluation in stroke survivors) was conceived, which has now been completed.

The rational basis of the ACCESS study was the question whether early treatment of elevated blood pressure values in stroke treatment using a substance which affects the renin-angiotensin system in a blocking manner, could reduce the morbidity, mortality and neurological deficits resulting from such strokes. The main question posed related to the use of early antihypertensive treatment of patients with elevated blood pressure values after a stroke.

It was unexpectedly found in an evaluation of clinical experiments with 342 enrolled patients who were treated with candesartan from 72 hours up to and including seven days after the stroke, in comparison to a placebo group which was first treated after one week, that there appeared a significant difference with respect to the cardiovascular end point in the course of a twelve month observation period in favor of the patients who received early treatment with Verum (17 vs. 30), which corresponded to a difference of about 43%. This difference was so marked that the recruitment of further patients was terminated on the advice of the safety committee.

Preferred substances for inhibiting the renin-angiotensin system in accordance with the invention include angiotensin-receptor blockers, ACE-inhibitors and vasopeptidase inhibitors. Especially preferred agents in this regard include candesartan, irbesartan, valsartan, losartan, telmisartan, eprosartan, lisinopril, quinalapril, benazepril, ramipril, perindopril, fosinopril and omapatrilate and their pharmaceutically acceptable salts and esters.

In accordance with the invention, the substance for inhibiting the renin-angiotensin system should be administered during the acute phase of the stroke, preferably from 0 to 72 hours, particularly preferably from 0 to 36 or 0 to 24 hours after the onset of the stroke up to and including at least seven days after the stroke. In accordance with the invention it has further been found that the substances for inhibiting the renin-angiotensin system exhibit protective effects which go beyond their blood pressure lowering affects both in the therapy of acute strokes (0 to 7 days after the stroke) and also in secondary prophylaxis, i.e., more than 7 days after the acute stroke.

In accordance with the invention the term “acute stroke” should be especially understood to include cerebral ischemia and especially preferably acute cerebral ischemia. It is particularly preferred to treat patients with elevated blood pressure, especially patients having a systolic blood pressure of more than 180 mmHg and/or a diastolic blood pressure of more than 105 mmHg.

After enrollment of the patients in the study, a randomized therapy with candesartancilexetil or placebo was carried out. Depending upon the measured blood pressure values and patient specific criteria, the therapy was begun with once daily administration of one half to one tablet (corresponding to from 4 to 8 mg candesartancilexetil or placebo). In cases of impaired swallowing ability, the administration can be effected through a stomach tube.

After the second day of therapy the dose can be increased in cases of insufficiently reduced blood pressure values (>160 mmHg systolic pressure and/or >100 mmHg diastolic pressure) to once daily administration of one tablet or once daily administration of two tablets (corresponding to 8 mg or 16 mg candesartancilexetil or placebo).

If this therapy is not sufficient, after the seventh day of therapy a combination therapy can be commenced at blood pressure values of >160 mmHg systolic and/or >100 mmHg diastolic pressure. The basis for the changes in therapy is the average value of at least three blood pressure measurements per day. Examples of suitable combination partners are listed hereinafter:

| | | | |
|---|---|---|---|
| Salureticum: | Hydrochlorothiazaide: | (e.g. Esiderix) | 12.5-25 mg |
| Calcium | Felodipine: | (e.p. Modip) | 2.5-5 mg |
| Antagonists: | | | |
| Betablocker: | Metoprolol: | (e.p. Beloc) | 50-100 mg |

The target blood pressure reduction amounted to 10-15% within 24 hours.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. A method of treating an acute stroke in a patient suffering therefrom, said method comprising administering to said patient a pharmaceutically effective amount of a substance which inhibits the renin-angiotensin system.

2. A method according to claim 1, wherein the inhibiting substance is an angiotensin-receptor-blocker, an ACE-inhibitor, or a vasopeptidase inhibitor.

3. A method according to claim 1, wherein the inhibiting substance is administered in combination with an antihypertensive agent.

4. A method according to claim 1, wherein the inhibiting substance is an angiotensin-receptor-blocker selected from the group consisting of candesartan, irbesartan, valsartan, losartan, telmisartan and eprosartan.

5. A method according to claim 1, wherein the inhibiting substance is an ACE-inhibitor selected from the group consisting of captopril, enalapril, lisinapril, quinalapril, benazelpril, ramipril, perindopril and fosinopril.

6. A method according to claim 1, wherein the inhibitor substance is the vasopeptidase inhibitor omapatrilate.

7. A method according to claim 1, wherein the inhibiting substance is candesartancilexetil.

8. A method according to claim 1, wherein said patient is suffering from a cerebral ischemia.

9. A method according to claim 1, wherein the treatment is initiated from 0 to 72 hours after onset of the acute stroke.

10. A method according to claim 9, wherein the treatment is initiated from 0 to 36 hours after onset of the acute stroke.

11. A method according to claim 1, wherein the patient is suffering from elevated blood pressure.

12. A method according to claim 11, wherein the patient has a systolic blood pressure of more than 180 mmHg or a diastolic blood pressure of more than 105 mmHg or both.

13. A method according to claim 1, wherein the treatment is continued for more than seven days.

14. A method of inhibiting a secondary stroke, said method comprising administering to a patient at risk for secondary stroke an effective amount of a substance which inhibits the renin-angiotensin system.
